(19) United States
(12) Patent Application Publication
(10) Pub. No.: US 2020/0363398 A1
(43) Pub. Date: Nov. 19, 2020

(54) PLATFORM FOR EARLY DETECTION OF PATHOGEN INFECTION

(71) Applicant: The Charles Stark Draper Laboratory, Inc., Cambridge, MA (US)

(72) Inventors: Else M. Vedula, Cambridge, MA (US); Kirsty A. McFarland, Melrose, MA (US); Amanda Nicole Billings-Siuti, Framingham, MA (US); Andrew P. Magyar, Arlington, MA (US)

(21) Appl. No.: 16/961,173

(22) PCT Filed: Jan. 9, 2019
(86) PCT No.: PCT/US2019/012835

§ 371 Date: Jul. 9, 2020

Related U.S. Application Data
(60) Provisional application No. 60/615,199, filed on Oct. 4, 2004.

(51) Int. Cl. G01N 33/50; C12Q 1/18

(52) U.S. Cl. CPC G01N 33/5038; G01N 2500/10; G01N 33/5014; C12Q 1/18

(57) ABSTRACT

A method for identifying an interaction between a pathogen and a biological agent includes providing a platform for supporting cell growth, seeding different interaction sites on the platform with different biological agents, perfusing the platform with a fluid that carries substances for promoting growth and maintenance of the cells, exposing all of the interaction sites to a solution containing viruses, and detecting evidence indicative of the interaction, the evidence comprising evidence indicative of a change in structure or composition of a medium at the interaction site. The biological agent includes cells alone or cells with another substance.

### RELATED APPLICATIONS

This application claims the benefit under 35 U.S.C. § 119(e) of U.S. Provisional Application No. 62/615,199, filed on Jan. 9, 2018, which is incorporated herein by reference in its entirety.

### FIELD OF INVENTION

The invention relates to methods of assessing biological interactions/associations between biological entities, such as cells and viruses, and in particular, to methods and systems for ascertaining interaction between a virus or cell and other biological agents, such as an antibody or a biological substance such as a toxin.

### BACKGROUND OF THE INVENTION

Quickly and accurately assessing interactions/reactions between cells, tissues, microbiological pathogens such as viruses and bacteria, toxins and other harmful environmental substances is of key importance in the world today. For example, screening to efficiently identify pathogenic microorganisms, or human exposure to a toxin and quickly test effective drugs, antibodies or antidotes is critically important to treat or prevent disease, especially when facing possible epidemics.

Naturally, to carry out these tasks on a small scale is fairly simple. One simply performs numerous experiments. However, such specific time point testing may not always capture the critical interactions between biological agents that are needed to accurately identify viable drugs or anti-toxins. Particularly important is the ability to screen interactions between biological agents such as cells and bacteria and anti-bacterial agents as they interact over a real time-line, not just at the particular point in time that a sample was obtained and tested.

Another important aspect of quick and efficient screening of biological interactions, for example, in detecting viral infections and antiviral drug or vaccine production, is a steady and consistent supply of viral particles. Thus, to make copious amounts of vaccine, one must have on hand a great many viruses.

In general, one cannot simply gather virus from the wild with great efficiency. Instead, it is more efficient to propagate viruses. This is typically carried out by finding a cell for the virus to infect. The cell will then lyse and produce more viruses, which then infect neighboring cells. This allows viruses to additionally reproduce and provides a rich source of viruses.

However, not just any cell will do. In many cases, a virus will not infect the cell. In other cases, the virus could kill the cell without replication. In either case, the result is the same: no viruses are produced. It is therefore important, if one wishes to produce a large number of viruses, to find a cell that the virus can use to replicate itself.

A similar problem arises in the production of antibodies against a particular virus or a particular toxin. Procedures for synthesizing different antibody molecules are well known. However, an antibody must be specific for a particular virus or toxin. Thus, some way must be found to efficiently determine which of a range of antibodies will be effective against a particular virus.

However, when the number of combinations becomes larger, the time required to carry out such screening becomes prohibitive. Given the rapid distribution of infectious agents in the modern world, a novel and particularly virulent virus may cause considerable depopulation before researchers have even made a dent in the required experimentation. Accordingly, it is no exaggeration to suggest that the ability to develop and produce antibodies and vaccines against viruses quickly may not be altogether unimportant to the survival of the human species.

### SUMMARY OF THE INVENTION

In one aspect, the invention features a method for monitoring or identifying a molecular or biological interaction or association between one, or more, biological entities or units (also referred to herein as “agents”). A biological “entity” or “unit” is defined herein as a cell(s) or cell(s) obtained from an organism (e.g., a mammal or human) or from an organism's tissue or blood (e.g., kidney tissue, whole blood or serum). A biological entity can also include a pathogenic entity, or a substance derived from, or produced by, a pathogenic entity. A pathogenic entity can be a microbial pathogen such as a virus, bacterium, fungus or any other pathogenic microorganism, For example, as described herein, an interaction can be a biological interaction between a viral pathogen and a biological agent/entity such as a cell or tissue. As another example, the biological interaction can be between a pathogenic substance such as a toxic substance, or toxin, produced by a microorganism (or from a plant) and a cell. Toxins can be an exotoxin or endotoxin (e.g., from Clostridium botulinum, Clostridium tetani, Bacillus anthraces, E. coli, or marine toxins from shellfish), and the biological interaction between the toxin and the cell may result in cell lysis. Toxins can also be chemically/synthetically produced as well as biologically or environmentally produced. In yet another example, the interaction can be between a virus, a cell and an antibody that neutralizes the virus and inhibits cell entry resulting in the inhibition of virus replication and cell lysis. Another example—is the interaction between a bacterium, human tissue, and an antibacterial drug that neutralizes bacterial virulence. In a further example, the interaction can be between a cell toxin, a cell and an antibody that neutralizes the toxic effect of the toxin and inhibits cell damage or lysis.

Following a biological interaction as described above, for example, of a virus, cell and antibody, and the identification of a neutralizing antibody, the method can continue to monitor the characteristics of the cell for regeneration and growth. For example, after the exposure of a cell to a toxin and candidate neutralizing antibody, and the confirmation that the antibody neutralizes the toxin, the cell can be monitored for characteristic activities or functions indicating cell survival or regeneration. Such characteristics are known to those of skill in the art.

Such a method includes providing a platform for supporting cell growth in high throughput (see for example, U.S. Pat. No. 10,018,620, and U.S. Application 2018/0142196, the teachings of all of which are herein incorporated by reference). Such a platform includes a plurality of interaction sites, comprised of substrates supportive of long term cell culture, controlled fluid delivery mechanisms, and the ability to monitor interaction between at least 2 biological entities. The interaction sites may be microfluidic in nature and comprise a 2D cell culture substrate or an architecture to encourage 3D formation of biological entity interaction. In one embodiment, the cell culture substrate could be a semi-permeable membrane. In another embodiment, the culture environment could be a 3D gel. Real-time monitoring of biological entity interaction can be non-destructive and multiplexed.

The method continues with the seeding of the different interaction sites with different biological agents. These biological agents include cells or cells in combination with other substances, such as antibodies. The platform is equipped to deliver controlled amounts of fluid flow for nutrient and oxygen perfusion to the biological agents. Then, the method continues with the perfusion of the platform with a fluid that carries substances for promoting growth and maintenance of the cells and exposure of all of the interaction sites to a solution containing bacteria or viruses, for example. This is followed by one, or more, means of detection of evidence indicative of the interaction or activity of interest. Such activity detection means can be, for example, integrated into the apparatus/platform for real-time, or substantially real-time, monitoring, or can be a subsequently performed assay apart from the platform for the detection of chemical or biological substances such as the expression of specific proteins. Importantly, the detectable activity provides evidence indicative of a change in composition or structure of a medium at the interaction site.

Additionally, suitable platforms for assessing such biological interactions can include micro-bead carriers, or other suitable materials to form scaffolds for cells, in particular adherent cells. Further, such scaffolds can be provided in droplets for microfluidic analysis.

In one embodiment of the present invention, the pathogens are selected to be microorganisms such as viruses, bacteria or yeast, and the medium is an intracellular medium, whereas in others, the medium is an extracellular medium.

In one embodiment, seeding the different interaction sites of the platform with different biological agents comprises seeding the interaction sites with different kinds of cells, for example those in which evidence indicates virus replication.

In another embodiment, seeding the different interaction sites of the platform with different biological agents comprises seeding the interaction sites with different kinds of antibodies and the same kind of cell. Among these are practices in which the evidence indicates that an antibody prevented infection of cells.

In yet other embodiments, seeding different interaction sites of the platform with different biological agents includes seeding the interaction sites with different kinds of antimicrobial agents and the same kind of cell.

Other embodiments of the present invention include those in which detecting evidence indicative of the interaction includes detecting evidence of virus replication and those in which detecting evidence includes detecting evidence indicative of antibody activity, such as antibodies preventing infection of cells.

Yet other embodiments include those in which detecting evidence indicative of the interaction includes detecting evidence of pathogen replication and those in which detecting evidence includes detecting evidence indicative of antimicrobial agent activity, such as antimicrobial agents preventing interaction between microbes and cells.

Other embodiments include those in which detecting evidence indicative of the interaction include detecting a change in metabolite level, those in which it includes detecting levels of an intracellular compound, those in which it includes detecting a level of an extracellular compound, those in which it includes detecting a level of a compound that is depleted during the course of pathogen formation, and those in which it includes detecting a level of a compound that is synthesized during the course of pathogen formation.

Some embodiments include those in which detecting evidence indicative of the interaction comprises detecting a glucose level.

In some embodiments, detecting evidence indicative of the interaction comprises detecting a change in metabolite level, an example of which would be a change in ATP level.

In other embodiments, detecting evidence indicative of the interaction comprises detecting a change in at least one of pH and pOH, or detecting evidence of occurrence of a redox reaction.

Yet other embodiments include those in which detecting evidence indicative of the interaction comprises optically detecting evidence of changes in aggregation of matter within a cell. Such changes are indicative of some kind of cellular change. In cases where a cell has been exposed to a pathogen, such a change could be evidence of pathogen formation. For example, in the case where the pathogen is a virus, such a change can be indicative of viral replication.

A variety of ways are available to detect such changes in aggregation. Among these are dynamic light scattering. A particularly useful method is to use angle-resolved low-coherence interferometry. This is particularly useful when observing backscatter in an optically complex environment.

Additional embodiments of the invention include those in which detecting evidence indicative of the interaction comprises using dynamic light scattering to detect evidence of virus formation, those in which it includes using an interferometer to detect evidence of virus formation, and those in which it includes using angle-resolved low-coherence interferometry to detect evidence of virus formation.

Some embodiments include the additional step of, based on the interaction, identifying a host for the virus.

Other embodiments include the additional step of, based on the interaction, identifying an antibody against the virus.

For example, seeding the different interaction sites of the platform with different biological agents comprises seeding at least one of the interaction sites with a plurality of antibodies and the same kind of cell. These examples can include the further step of identifying which of the antibodies is effective at blocking infection. A suitable method for doing so includes carrying out a binary search.

Other examples include, after having identified the interaction, identifying a pathogen that engaged in the interaction and seeding a bioreactor with that identified pathogen.

Also examples are those that include, after having identified the interaction, identifying a biological agent that engaged in the interaction and producing additional amounts of said biological agent.

In another aspect, the invention features an apparatus/device comprising a platform and a fluid delivery system that is coupled to the platform for providing an environment conducive to cell growth and maintenance on the platform. The platform comprises one, or more, interaction sites that are separated from each other. An activity detector is used to monitor the specific interaction(s) at the one, or more sites. The activity detector can be coupled to, or integrated into, the apparatus or platform.

Alternatively, the activity detector can comprise a detection means separated from the apparatus/platform, wherein the activity detector comprises means for performing one, or more, biochemical assays suitable for specifically detecting the desired interaction. In this embodiment, the detection can comprise, for example, obtaining a sample (e.g., removing a sample of supernatant from a reaction well or channel at a specific reaction site) and assaying the sample (in real-time or later) with a suitable chemical or biological assay. In either optional embodiment, the activity detector detects evidence of an interaction between a biological agent and a pathogen at the interaction site. The evidence includes a change in either structure or composition of a medium at the interaction site, and can include, for example, transepithelial electrical resistance (TEER) or biochemical assessment of expressed or suppressed substances such as cytokines.

In some embodiments, the activity detector comprises an interferometer. Among these are interferometers that provide an angular distribution of light that has been back-scattered from the interaction site. This light can then be used in connection with obtaining structural information about subsurface layers. Also among the embodiments are those in which the interferometer is detector is configured to carry out angle-resolved, low-coherence interferometry.

Additional embodiments include those in which the activity detector provides data representative of dynamically scattered light to a processor that recovers, at least in part on the basis of the data, information indicative of a change in structure at the interaction site.

In some embodiments, the activity detector detects a change in chemical composition. Among these are activity detectors that detect a change in a concentration of metabolite at the interaction site, or a change, at the interaction site, of a concentration of a substance. An example of such a change is a change in glucose levels at the interaction site.

Other embodiments include those in which the activity detector detects a change in an ion concentration at the interaction site, or evidence of a redox reaction at the interaction site, or a change in an electrical property of a medium at the interaction site.

These and other features will be apparent from the following detailed descriptions and the accompanying figures.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. The patent or application file contains at least one drawing executed in color. Of the drawings:

FIG. 1 is a schematic view of an apparatus for identifying an interaction between a pathogen and a biological agent; and

FIG. 2 shows a method for using the apparatus shown in FIG. 1.

FIG. 3 depicts the experimental setup using four uropathogenic E. coli strains with the renal proximal tubule kidney tissue model, assessed in quadruplicate at MOI of 10 and 100, after 1 h and 8 h, using a total of 64 out of the 96 available interaction sites. Another 16 sites were used as negative controls, using a total of 80 interaction sites out of 96.

FIG. 4A-D are photomicrographs depicting the results of immunofluorescence detection at the 1 hour timepoint.

FIG. 5A-D are photomicrographs depicting the results of immunofluorescence detection at the 8 hour timepoint.

FIG. 6 A-B depict transepithelial electrical resistance (TEER) measurements recorded throughout the experiment on days 2, 5, 6, 7 and post-inoculation.

FIG. 7A-B depict cytokine levels as compared to the no bacteria control at 8 hours post-inoculation.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

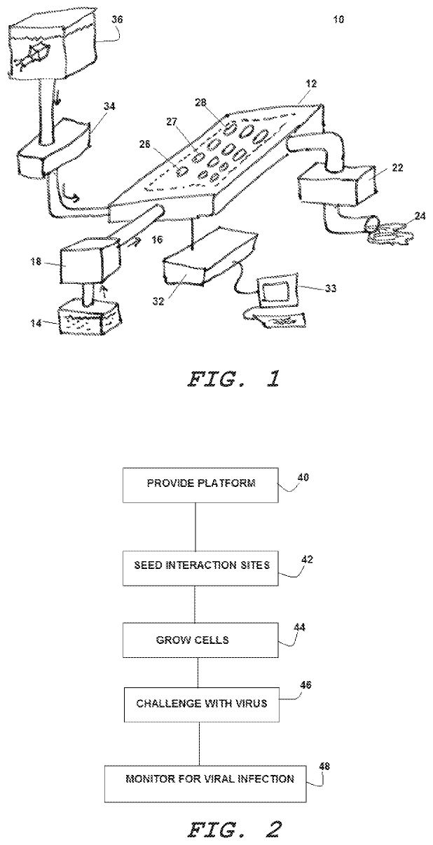

FIG. 1 shows a schematic diagram of an apparatus 10 for detecting interaction between a pathogen and a biological agent in a high throughput manner. In the particular embodiment shown, the pathogen is a virus. However, the apparatus and methods described herein remain essentially unchanged when different kinds of pathogens are used.

The apparatus 10 is intended to maintain living cells on a platform 12 for extended periods. As such, the apparatus 10 includes, in addition to the platform 12, a source 14 of an input solution 16 that contains nutrients and other factors for promoting cell growth and maintenance, as well as factors that promote robust infectivity, such as bile, mucins, or trypsin.

A first pump 18 pumps this input solution 16 through the platform 12. As the first pump 18 pumps the input solution 16 into the platform 12, a second pump 22 pumps an output solution 24 out of the platform 12. This output solution 24 includes waste products of cellular metabolism. A suitable implementation of an apparatus 10 that can be used to maintain cells in a platform is a microfluidic system such as that disclosed in U.S. Pat. No. 10,018,620 and U.S. Patent Application 2018/0142196, the contents of which are herein incorporated by reference.

The platform 12 is divided into different distinct interaction sites 26, 27, 28 that are isolated from each other and designed to support prolonged cell culture, the controlled delivery of infectious agents, and the monitoring of the biological entity interactions. These interaction sites 26, 27, 28 define an interaction array 30. Although only a few such interaction sites 26, 27, 28 are shown, this is only to avoid visual clutter in the drawing. In practice, practical considerations will limit number of such interaction sites 26, 27, 28. However, in one preferred embodiment, there are ninety-six such interaction sites 26, 27, 28 per platform 12.

The illustrated platform 12 supports multiple growth formats. Some examples of such growth formats include growth at an air-liquid interface, growth of immersed cell monolayers, and even growth of suspended cells that do not require any surface attachment.

The apparatus 10 further includes an activity-detector 32 in communication with a data-processing system 33. The activity-detector 32 obtains evidence indicative of the occurrence of an interaction between the viruses and biological agents that are disposed within the platform 12. The nature of the activity-detector 32 and precisely how these interactions are detected are both discussed below in detail.

In general, to identify an interaction between a virus and biological agent, the different interaction sites 26, 27, 28 are first seeded with different biological agents. Once the interaction sites 26, 27, 28 have been seeded, they are exposed to a pathogen solution 36. Such exposure can be carried out by a third pump 34 that pumps a pathogen solution 36 through the platform 12. This floods the interaction array 30 and thus exposes the biological agents within the interaction sites 26, 27, 28 to a suitable concentration of the pathogen. Alternatively, a pipette, or several pipettes in parallel, can drop pathogen solution 36 onto each interaction site 26, 27, 28. In the illustrated embodiment, the pathogen is a virus.

To identify a host that is suitable for viral production, the platform 12 is seeded with different candidate host cells at different interaction sites 26, 27, 28. As a result, each interaction site 26, 27, 28 will have a different kind of cell growing within it. The first and second pumps 18, 22 operate until cell growth is suitably established. They continue to operate throughout the procedure. In some embodiments, the interaction sites lie on a first side of a semi-permeable membrane while the first and second pumps 18, 22 move fluid along a second side of the membrane. In such embodiments, the cells and the virus lie on the first side of the membrane.

As time passes, there will be different outcomes at different interaction sites 26, 27, 28. For example, at the first interaction site 26, the virus may have no interaction with the cells. At the second interaction site 27, the virus may infect the cell but fail to replicate. However, at the third interaction site 28, the virus may infect the cell and replicate successfully. At such an interaction site 28, infected cells lyse, thus releasing new viruses that can then infect neighboring cells, thus initiating a chain reaction. It is these cells that can be used for making larger quantities of viruses, which can then be used for vaccine production.

In principle, one could wait to see if there are visible indicia of lysing followed by reinfection. This can be detected macroscopically by observing holes in a plaque formed by the growing cells. This procedure, which relies on observing changes in cell population, is a time-consuming procedure. The activity-detector 32 accelerates this procedure by observing changes in structure and/or composition that are caused by infection. Such observations can detect infection much faster than observations based solely on cell population, and can in fact observe the infection process almost in real time.

In some embodiments, the activity-detector 32 observes a change in the way the medium at the interaction site 26, 27, 28 scatters light. Other embodiments use interferometry to detect changes in interference patterns that result from, for example, the presence of additional particles, such as virus particles. These embodiments provide a way to infer and even map the existence of small particles, such as viruses.

In other embodiments, the activity-detector 32 observes a change in the chemical composition of the solution at the interaction site 26, 27, 28. For example, it is possible to observe changes in concentration of ATP or other metabolites. This can be carried out using colorimetric, fluorescence, or luminescence assays.

For example, one, or more of the biological or chemical entities comprising the interaction to be monitored can be detectably labeled with fluorescent tags or dyes. Such fluorescent tags are suitable to specifically detect the interaction to be detected/followed and can be used along with an optical readout means (either integrated into the apparatus/platform or apart from the apparatus/platform) to track the interaction in real-time through readout of fluorescent intensity and/or lifetime. This embodiment provides a platform where tracking can occur at the rates required due to the close integration of the electronics as well as the use of fast LEDs and fast photodiodes instead of lamps and cooled CCD cameras.

A method for detecting which of several antimicrobial agents is effective against a particular pathogen can be carried out in an analogous manner.

In those cases in which the pathogen is a virus and the antimicrobial agent is an antibody, it is possible to identify which of several antibodies is effective against a particular virus. This is carried out by seeding the platform 12 with different biological agents at different interaction sites 26, 27, 28. In this case, the biological agent consists of a cell and a candidate antibody. The cell is one that is known to be a suitable host for the virus in question. A suitable procedure for identifying such a cell has already been described above.

The candidate antibodies differ from one interaction site to the next, but the cell type remains constant. The first and second pumps 18, 22 operate until cell growth is suitably established and continue to operate throughout the procedure.

Once the population of cells is sufficient, the cells are exposed to a pathogen solution 36. Such exposure can be carried out by using a third pump 34 to flood the platform 12 with the pathogen solution 36. Alternatively, one or more pipettes can be used to drop pathogen solution 36 at each interaction site 26, 27, 28.

As time passes, there will be different outcomes at different interaction sites 26, 27, 28. For example, the antibody that is present at the first interaction site 26 may fail to prevent infection. Thus, the cell will lyse and spread infection. On the other hand, the antibody present at the second interaction site 27 may be just right for preventing infection by that virus.

The activity-detector 32 can thus be used in a manner similar to that already described to provide early-detection of successful replication. This provides a basis for rapidly assessing effectiveness of particular antibodies.

To identify which of several antimicrobial agents is effective against a particular pathogen, the platform 12 is seeded with different biological agents at different interaction sites 26, 27, 28. In this case, the biological agent consists of a cell and a candidate antimicrobial agent. The cell is one that is known to be susceptible to being harmed by the pathogen in question. A suitable procedure for identifying such a cell can be readily adapted based on what has already been described above.

The candidate antimicrobial agents differ from one interaction site to the next, but the cell type remains constant. The first and second pumps 18, 22 operate until cell growth is suitably established and continue to operate throughout the procedure.

Once the population of cells is sufficient, the cells are exposed to a pathogen solution 36. Such exposure can be carried out by using a third pump 34 to flood the platform 12 with the pathogen solution 36. Alternatively, one or more pipettes can be used to drop pathogen solution 36 at each interaction site 26, 27, 28.

As time passes, there will be different outcomes at different interaction sites 26, 27, 28. For example, the antimicrobial agent that is present at the first interaction site 26 may fail to prevent harm to the cell. On the other hand, the antimicrobial agent present at the second interaction site 27 may be just right for preventing harm to the cell from that pathogen.

In the above example, the interaction between the pathogen and the cell that ultimately harms the cell is expected to lead to both structural changes and changes in composition.

Examples of structural changes include changes in the aggregation of matter at the interaction site. Examples of changes in composition include changes in metabolite levels or changes in substances that are depleted or produced during replication.

Such changes may be in the intracellular medium or in the extracellular medium in the vicinity of the cell. In either case, the structural changes or changes in composition can offer a clue to the fact that an antimicrobial agent has failed to prevent a pathogen from interacting with a cell.

The activity-detector 32 detects such changes. The activity detector can be integrated into the apparatus. Alternatively, the activity detector can comprise a separate means or device for a chemical or biological substance detection assay performed apart from the apparatus and the results correlated with the specific interaction point as well as the specific time-point. Such chemical and biological assays are well-known to those of skill in the art. In particular, a cytokine expression profile panel can comprise a biological assay as described in the Exemplification herein.

In particular, the activity-detector 32 can be used in a manner similar to that already described to provide early-detection of cellular harm caused by pathogens. This provides a basis for rapidly assessing effectiveness of particular antimicrobial agents. To further accelerate the screening procedure, it is possible to seed each interaction site 26, 27, 28 with a biological agent that consists of host cells and an antibody cocktail having a mixture of antibodies. In that case, if the cells in a first interaction site 26 die, one can infer that none of the antibodies in that first interaction site 26 were effective. If the cells in a second interaction site 28 live, one can infer that at least one of the antibodies in the antibody cocktail was effective. This method can also be used to identify cytotoxicity of antibody cocktails.

The use of antibody cocktails in an interaction site 26, 27, 28 instead of individual antibodies provides a way to quickly eliminate large numbers of antibodies from further consideration. Through a binary search, it becomes possible to quickly hone in on the one or more antibodies that turned out to be effective at the second interaction site 28. This binary search process likewise significantly accelerates the process of identifying the antibodies that are effective against the virus in question.

FIG. 2 shows steps in a process carried out by the apparatus of FIG. 1 to identify an interaction between a microbe and a biological agent. The process begins with providing a platform 12 for supporting cell growth (step 40). The platform 12 includes a plurality of interaction sites.

Although these interaction sites 26, 27, 28 are described as being arranged as rows and columns of a rectangular interaction array 30, this particular arrangement is by no means required. What is required instead is a way to encode the identity of a biological agent at a particular interaction site 26, 27, 28. In a case in which the interaction sites 26, 27, 28 are fixed relative to some frame-of-reference, as shown in FIG. 1, a convenient way to encode this information is by the spatial position of the interaction site 26, 27, 28 in some coordinate system. Arranging interaction sites 26, 27, 28 in an interaction array 30 makes this particularly convenient.

The process continues with seeding the different interaction sites 26, 27, 28 with different biological agents (step 42) that comprise cells. In some practices, the biological agent includes more than just cells. For example, a biological agent may be a combination of cells and antibodies, or a combination of cells and an antibody cocktail.

To promote a healthy population of living cells, the process includes causing an input fluid to perfuse through the platform 12 (step 44). Typically, the input fluid will contain nutrients and any other factors needed to promote cellular growth and maintenance.

Once seeded, the cells typically achieve confluency within twenty-four or forty-eight hours. At this point, the process continues with a viral challenge (step 46). This permits interaction between the viruses and the various biological agents distributed among the interaction sites 26, 27, 28.

As time lapses, the platform 12 is monitored in real time for signs of such interaction. Eventually, evidence of such interaction is detected (step 48). Examples of such evidence include a change in composition of the medium within the interaction site.

Several embodiments of the activity-detector 32 are available, depending on the physical properties to be monitored. These include activity-detectors 32 that monitor impedance, transepithelial electrical resistance, glucose demand, acidity, alkalinity, and occurrence of redox reactions. Additional embodiments of activity detectors 32 include those that carry out biochemical assays of substances present in the interaction site 27 and optical assessments of cell morphology or intracellular activity at the interaction site 27. An activity-detector 32 can also be configured to monitor more than one of the foregoing parameters rather than relying on only one of them.

Among the most useful activity detectors 32 are those that carry out metabolic assays for detecting early-stage infection across many classes of viruses and types of cells. Such assays are calibrated with baseline viral-infection screening data. The generation of such baseline screening data would include measuring a host cell's phenotype during the course of an infection cycle. Such data often reveals a well-defined point in time at which one can safely say that there has been a virus-mediated change to the phenotype.

Following the viral challenge (step 46), such an activity detector 32 monitors the challenged cell's phenotype in an effort to detect the occurrence of this point. Reliance on metabolic changes caused by infection permits such an activity detector 32 to detect the change early in the infection process. This provides a basis for obtaining a prompt indication of viral infection.

Real-time monitoring of metabolite level is particularly useful because viral infection changes the host's cellular metabolism in a manner that promotes viral replication. Since viral replication requires additional energy, and since energy production is linked to metabolism, a sudden demand for energy will make itself apparent through a corresponding change in metabolism. In particular, the sudden demand for energy that arises from replication manifests itself in increased production and/or depletion of ATP. Thus, a sudden change in ATP concentration serves as a marker for viral replication.

In some embodiments, an activity detector 32 that monitors metabolic activity monitors cellular ATP concentration using a luminescence-based assay. In such an assay, the luminescent signal is proportional to the concentration of ATP. Such an assay is also amenable to high-throughput screening.

A particular advantage arises because it is possible to detect a change in ATP concentration long before any cytopathic effects become apparent. As a result, real-time monitoring of ATP concentration permits detection of viral replication well in advance of the cytopathic effects that would normally announce such replication. In fact, an ATP-luminescence phenotype screen may allow what is effectively real-time detection of viral infection. This significantly accelerates the screening process.

An activity detector 32 that implements an ATP-luminescence phenotype assay offers considerable sensitivity. In many cases, such an activity detector 32 is capable of measuring changes in cellular metabolism even when the number of cells is below the detection limits of standard fluorometric assays.

Since viral infection modifies host cellular metabolism, an activity detector 32 can instead implement an alternative metabolic assay to determine whether viral infection has occurred. For example, in some embodiments, the activity detector 32 implements a fluorometric water-soluble redox indicator. Other methods could be used to detect viral infection, including standard cell viability or cytotoxicity assays that depend on cytopathic effects, such as lactate dehydrogenase release or live-dead staining.

Other embodiments of the activity detector 32 monitor changes wrought by virus replication to optical properties of a medium. As a virus grows, certain biomolecules will be synthesized within a cell. These biomolecules will eventually assemble or aggregate to form a whole infectious virus. As this aggregation occurs, it leaves behind certain subtle clues. In particular, the aggregation locally modulates the refractive index in the cell and its surroundings.

In some embodiments, the activity detector 32 directly detects this virus-induced modulation optically using a device that comprises a light source, such as a diode or laser, optics for focusing or shaping the light, and a detector or camera that interfaces with the platform 12. Such activity detectors 32 include optical devices, such as fiber-optical or free-space optical devices, that directly characterize the growth of a virus at very early stages.

In some embodiments, the activity detector 32 carries out interferometric measurements of the amplitude of the scattered light field. This is particularly useful for detecting particles that have a low refractive-index, such as viruses in aqueous solution.

In other embodiments, the activity detector 32 carries out angle-resolved, low-coherence interferometry. Such an activity detector 32 measures angular distributions of back-scattered light and uses it to recover structural information about subsurface layers.

In yet other embodiments, the activity detector 32 relies on optical diffraction tomography. Such an activity detector 32 includes a Mach-Zehnder interferometer that characterizes complex optical fields. The processor 33 uses this amplitude and phase delay information to reconstruct a three-dimensional map of the cell showing the modulation of refractive index at various locations within the cell.

Other embodiments of the activity-detector 32 rely on nanoparticles, fluorescent, luminescent, or colorimetric dyes to infer infection-induced changes in such features as a change in the potential across the membrane upon which the cells grow, a change in a particular metabolite concentration, a change in the concentration, amount, or identity of any one of a variety of biomolecules, including proteins and nucleic acids.

In other embodiments, the platform 12 includes electrical or electrochemical probes integrated therein. Such probes can be used to measure infection-induced changes in electrochemical properties of the medium. Observable properties that can change as a result of infection include pH and pOH and oxygen concentration. Such probes are also useful for direct measurement of evidence of occurrence of redox reactions that may accompany infection. From such measurements, it is possible to infer the existence of free radicals and reactive oxygen species. Such probes also make possible the observation of enzymatic transduction to provide redox-based detection of small molecules, metabolites, and other biomarkers that may be indicative of infection.

The use of multiple sensors and assays, together with the large number of interaction sites 26, 27, 28 provides a rapid way to identify viral infection and replication independently of plaque assays.

Upon identification of cell type for viral amplification, it becomes possible to adapt process protocols and small-scale stir-perfusion and rotating vessel bioreactors with micro-carriers to produce sufficient quantities of viral particles for all downstream platform applications, including animal studies. An example of a suitable micro-carrier is that sold under the name CYTODEX® by GE Biologics.

### Exemplification: Acute Pyelonephritis Model

Fimbriae are surface-expressed appendages that mediate bacterial adherence to host cells and tissues. P fimbriae (encoded by the pap genes) of uropathogenic E. coli (UPEC) are the major virulence factor influencing pyelonephritis, or infection of the upper urinary tract by UPEC. P fimbriae specifically interact with glycolipids that are expressed by erythrocytes and host kidney cells (Mulvey et al., 2000), and their attachment to host cells aids bacteria in withstanding the flow of urine. UPEC also express Type 1 fimbriae (encoded by thefim genes), which have specificity for mannosylated glycoproteins, and are generally considered more important in the initial urinary tract infection of the bladder. Some studies have suggested that Type 1 fimbriae may also play a role in upper urinary tract colonization despite the lack of mannosylated receptors on renal epithelia. It has been suggested that in the presence of fluid flow in the proximal tubule of a living kidney, both P and Type 1 fimbriae act synergistically to promote epithelial and inter-bacterial interactions, respectively, to withstand flow and enhance colonization (Melican et al., 2011). Host immune response to bacterial urinary tract infections also appears to be influenced by fimbrial adhesion, which is believed to bring the bacterial endotoxin (lipopolysaccharide) into proximity with host cells, strongly inducing cytokine expression.

Model systems of UPEC infection of the kidney typically use murine models or in vitro human monolayer cultures. Many animal models do not possess the same receptor proteins required for colonization of human cells, nor the same cytokines. In the present experiment a human organ system (see e.g., U.S. Pat. No. 10,018,620, the teachings of which are incorporated by reference in their entirety) was used to assess the biological interactions/associations of UPEC bacteria in an in vitro model of human kidney proximal tubule infection—acute bacterial pyelonephritis. The platform enabled testing of multiple fimbrial mutants of UPEC clinical isolates, in a more physiologically-relevant human tissue model, and in the presence of relevant fluid flow conditions experienced by the host cells and the infecting bacteria in the human renal proximal tubule. Knowledge for culturing of the required cells within the device is known to those of skill in the art. Multiple bacterial strains, with different fimbrial expression profiles, that have been clinically well characterized were available for testing. Fimbrial adhesion by UPEC is influenced by flow—a controllable feature of the device as described herein—enabling testing of the contribution of the two fimbrial types (P fimbriae, and Type 1 fimbriae) to colonization in the presence of flow. Numerous clinical and/or phenotypic markers are known for both the host and pathogen, allowing us to make testable hypotheses. Taken together, these factors allowed the testing of the utility of the device and its physiological accuracy, to assess host-pathogen interactions in a well understood human tissue model as described above.

Methods

Human Renal Proximal Tubule Tissue Model

Kidney cells were co-cultured in cell culture devices as described in Vedula et. al., 2017; U.S. Pat. No. 10,018,620; and U.S. Patent Application 2018/0142196. First, plates were treated to permit growth of the cells. Human microvascular endothelial cells (hMVECs) were seeded in basal channels, such that the cells adhered to the membrane that separates the basal and apical channels of the device. Two days later, the renal proximal tubular epithelial cells (RPTECs) were seeded in the apical channel, such that the cells may adhere to the membrane that separates the basal and apical channels of the device. Cells were counted to estimate loads prior to inoculation by the bacterial strains. Cells were grown under fluid flow of 10 ul/min, which is comparable to that experienced in the kidney tubule (Vedula et al., 2017).

Uropathogenic E. coli

The uropathogenic E. coli (UPEC) strains below were cultured by growth in LB agar plates, or in LB broth, using standard methods. Adherence phenotypes were verified using erythrocyte agglutination tests prior to inclusion in the study.

| Uropathogenic | | Adherence |
|---|---|---|
| E. coli strain | Genotype | phenotype |
| CFT073 | Wild type | Adherent |
| | clinical isolate | |
| UPEC76 | Δpap | No P fimbrial |
| | | adhesion |
| UPEC76 Δfim | Δfim Δpap | No P or |
| | | Type 1-fimbrial |
| CFT073_{OFF} | fim-OFF | Hyperadherent |
| | | (increased P |

To enable visualization of the microbes by immunofluorescence (IF) staining, all strains were made competent and transformed with the plasmid, pRudolph, that constitutively directs expression of red fluorescent protein (RFP). The transformed bacteria were selected for on carbenicillin, but were shown to maintain the plasmid over a 24 h period even in the absence of selective pressure: this was confirmed throughout the experiment by observation of identical CFU counts on LB agar with and without carbenicillin supplementation.

Infection Assays

Overnight cultures of the strains were measured by optical density at 600 nm, diluted in RPTEC growth medium to obtain specific multiplicity of infection (MOIs) ratios of 10 or 100 bacteria per host cell, and verified by CFU counts. Bacteria were introduced directly into the RPTEC growth channel and flow of 10 ul/min resumed.

Transepithelial Electrical Resistance (TEER)

TEER is a quantitative measurement of barrier function and/or tight junction formation of cells in culture, which was used to determine whether barrier function or cell integrity was damaged as a result of the bacterial infection, via host cell lysis or exfoliation. Using a proprietary device and method, TEER was assessed throughout the experiment. The device was sterilized after all TEER readings.

Immunofluorescence (IF) Labeling

To visualize the tissue markers, the device contents were fixed, permeabilized and blocked as described in Vedula et al., 2017. Primary antibody (mouse anti-ZO-1), to detect the tight junction protein marker of RPTECs, was added and fluorescent anti-mouse secondary antibody used to allow visualization. Host cell nuclei were stained using Hoechst. Bacteria were fluorescent due to constitutive expression of RFP.

Cytokine Expression Profiling

To measure the secretion of inflammatory cytokines in response to bacterial infection, supernatants were harvested from the devices, filter sterilized, and stored at −80. Analysis was performed using a custom panel to determine the concentration of the following cytokines by Luminex assay:

Interleukin-6 (IL-6): Pro-inflammatory cytokine; upregulated in UPEC infection (Frendeus et al., 2001)

Interleukin-8 (IL-8): Chemotaxis of neutrophils to infection site; upregulated in UPEC infection (Agace et al., 1993; Frendeus et al., 2001)

Monocyte chemoattractant protein 1 (MCP-1): Recruits monocytes, T cells, etc. to inflammatory sites caused by injury or infection (Su et al., 2014)

Interferon gamma (IFN-γ): Involved in immunity against viral and some bacterial infections (Khalil et al., 2000)

Tumor necrosis factor alpha (TNF-α): Expected to be secreted in response to bacterial LPS (Su et al., 2014)

Results

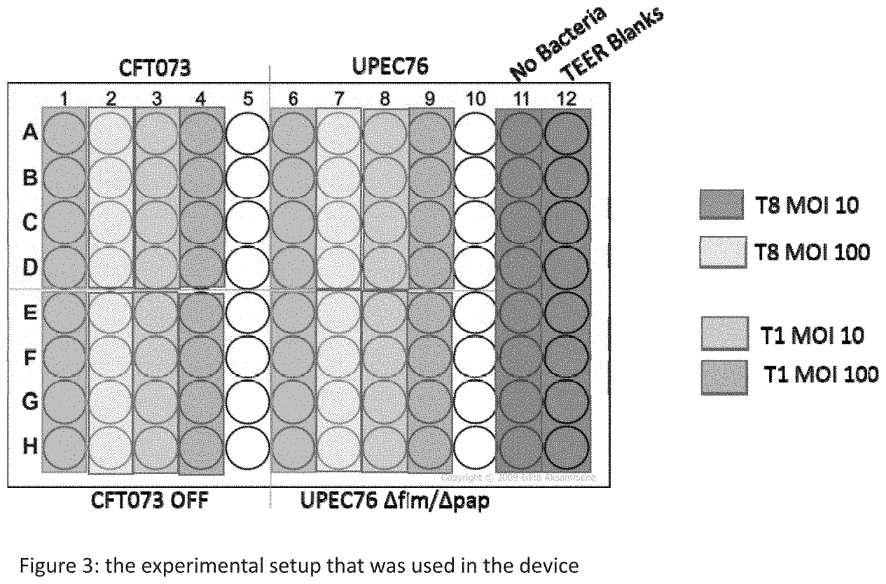

Interaction of the four listed UPEC strains with the kidney tissue model was assessed in quadruplicate at MOI of 10 and 100, after 1 h and 8 h, using a total of 64 out of the 96 available devices. Another 16 devices were used as negative controls, using a total of 80 devices out of 96 (FIG. 3)

Samples were harvested at 1 h and 8h post-inoculation for CFU counts. It was determined that bacterial counts did not change between strains, indicating that all four strains survived equally well, although a significant drop in CFUs were observed over 1-8 h in all strains. CFU counts were identical on selective or non-selective media from all samples tested, suggesting that the RFP-expression plasmid pRudolph was maintained even without selective pressure. Additionally, no significant difference was observed in CFU counts between the four strains grown only in RPTEC medium over a 24 h time course, indicating that strains had neither a growth advantage nor defect in RPTEC media.

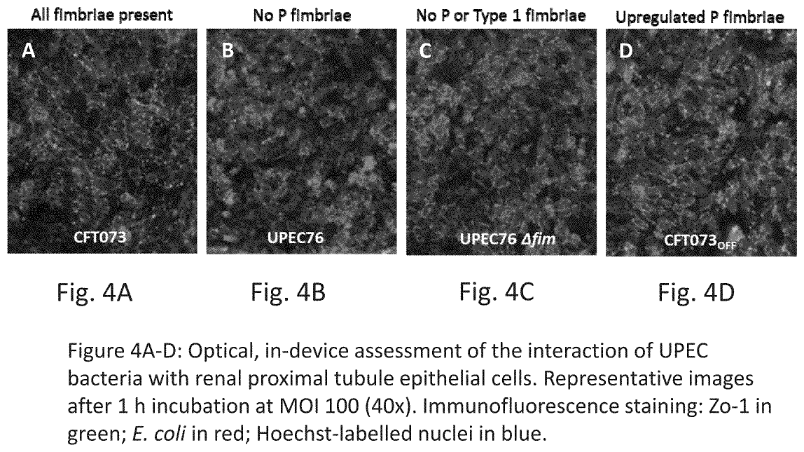

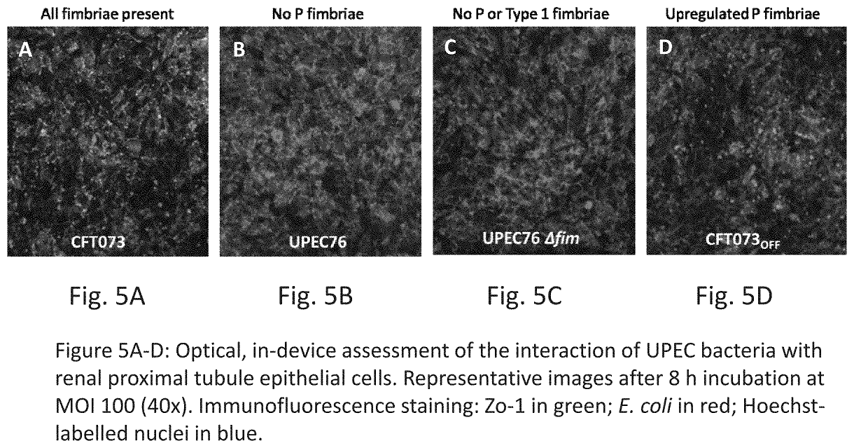

Immunofluorescence microscopy indicated that CFT073 and CFT073OFF were highly adherent to the RPTECs at both timepoints tested, whereas the UPEC76 strain (Δpap) adhered less well. UPEC76 Δfim appeared to be unable to colonize the RPTECs. Representative images taken at the 1 h and 8 h timepoints are shown in FIGS. 4A-D and 5A-D, respectively. Together, these data suggest that P fimbriae strongly influence colonization of RPTECs by clinical uropathogenic E. coli isolates, but suggests that Type 1 fimbriae may also play a role in adherence and colonization of RPTECs. These data are in agreement with research that suggests that Type 1 fimbriae may also play a role in urinary tract infections, but to a lesser degree than the P fimbriae, which are considered to be the major virulence factor in UPEC bacteria.

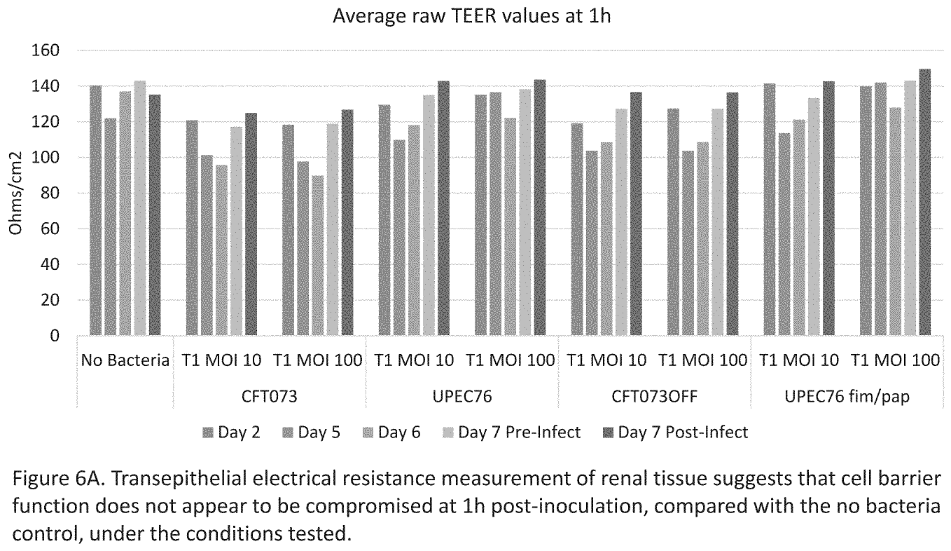

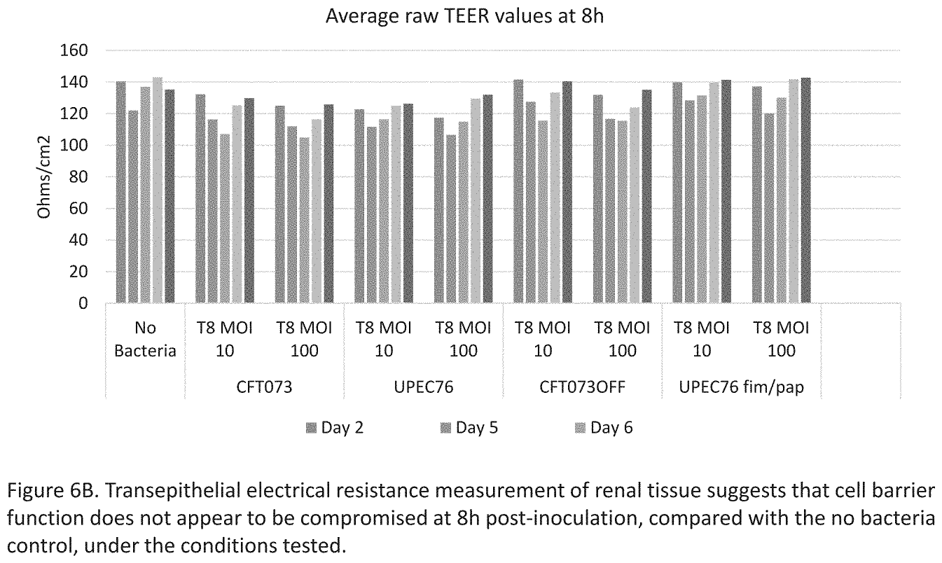

TEER measurements were recorded throughout the experiment on Days 2, 5, 6, 7 pre- and post-inoculation (FIG. 6A-D). Bladder cell exfoliation is known to occur in mice as a host defense mechanism against UPEC adhesion via Type 1 fimbriae (Mulvey et al., 2000). TEER measurements did not appear to significantly change over the course of our experiment, suggesting that the proximal tubule tissue barrier was not compromised under any of the conditions or with any of the strains, regardless of fimbrial phenotypes.

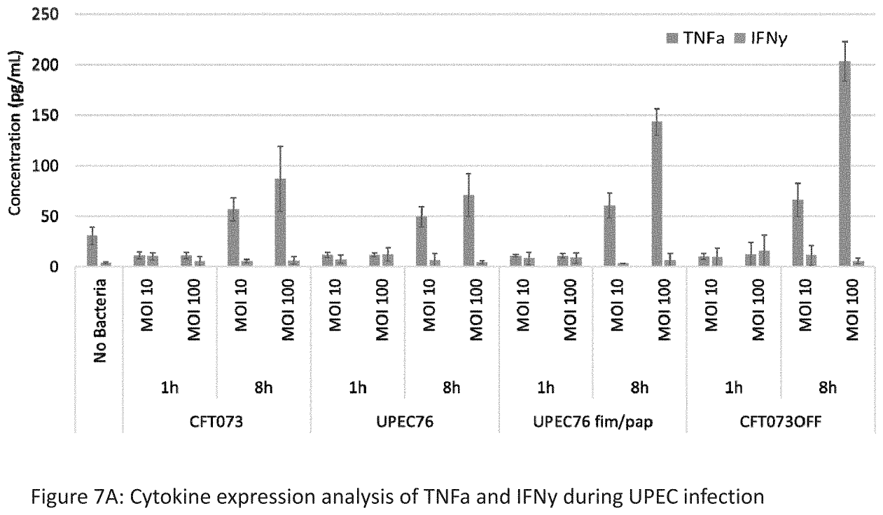

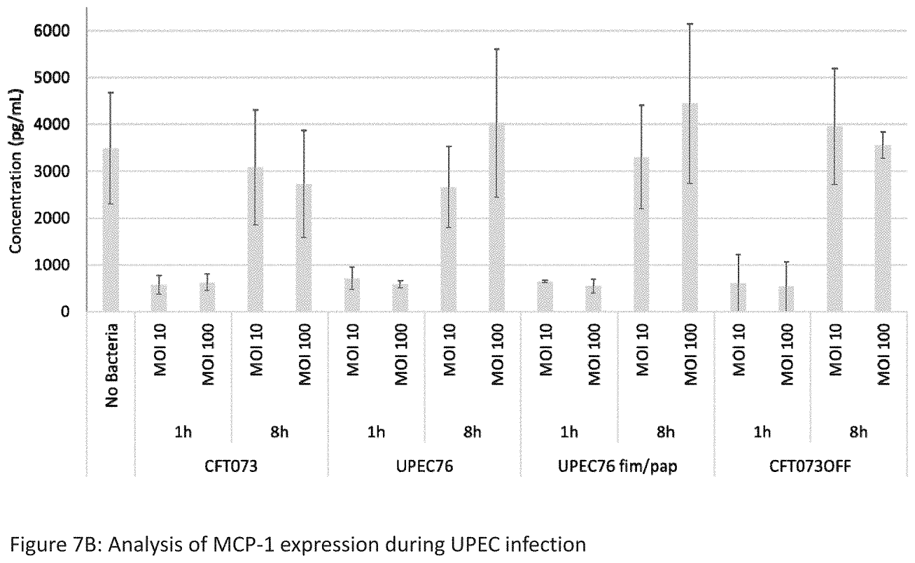

Cytokine expression tests indicated that the kidney tissue model was able to respond to the presence of all bacterial strains. Importantly, only cytokine concentrations from the 8 h ‘no bacteria’ control sample were determined in this assay. IL-6 and IL-8 levels were very strongly upregulated to levels that were above the detection range of the assay that was used (data not shown). Strong induction in IL-6 and IL-8 was an expected result that has consistently been reported in clinical testing of UPEC bacteria (Frendeus et al., 2001; Agace et al., 1993) but is suggested to require adhesion via P or Type 1 fimbriae (Frendeus et al., 2001; Hedlund et al., 2001). Due to inability to quantify the concentration in the samples, we are unable to determine whether the IL-6 or IL-8 concentrations displayed fimbrial-dependent differences, as all strains induced these cytokines to levels over the detection range, regardless of the strain adherence phenotype. TNF-α levels appeared to increase compared to the no bacteria control, and may vary depending on the strain and MOI, although this would need to be repeated for statistical significance (FIG. 7A). IFN-γ and MCP-1 levels did not appear to be upregulated in response to UPEC bacteria, as levels did not significantly increase over the no bacteria control (FIGS. 7A, and 7B).

### REFERENCES

Vedula E M, Alonso J L, Arnaout M A, Charest J L. A microfluidic renal proximal tubule with active reabsorptive function. PLoS ONE 12(10), 2017.
U.S. Pat. No. 10,018,620.
U.S. Patent Application 2018/0142196.
Mulvey M A, Schilling J D, Martinez J J, Hultgren S J. Bad bugs and beleaguered bladders: interplay between uropathogenic Escherichia coli and innate host defenses. Proc Natl Acad Sci USA. 97(16), 2000.
Melican K, Sandoval R M, Kader A, et al. Uropathogenic Escherichia coli P and Type 1 fimbriae act in synergy in a living host to facilitate renal colonization leading to nephron obstruction. PLoS Pathog. 7(2), 2011.
Agace W W, Hedges S R, Ceska M, Svanborg C. Interleukin-8 and the neutrophil response to mucosal gram-negative infection. J Clin Invest. 92(2), 1993.
Khalil A, Tullus K, Bartfai T, Bakhiet M, Jaremko G, Brauner A. Renal cytokine responses in acute Escherichia coli pyelonephritis in IL-6-deficient mice. Clin Exp Immunol. 122(2), 2000.
Su, X. et al. LRRC19 expressed in the kidney induces TRAF2/6-mediated signals to prevent infection by uropathogenic bacteria. Nat. Commun. 5, 2014.
Frendéus B, Wachtler C, Hedlund M, Fischer H, Samuelsson P, Svensson M, Svanborg C. Escherichia coli P fimbriae utilize the Toll-like receptor 4 pathway for cell activation. Mol Microbiol. 40(1), 2001.
Hedlund, M., Frendéus, B., Wachtler, C., Hang, L., Fischer, H., Svanborg, C. Type 1 fimbriae deliver an LPS- and TLR4-dependent activation signal to CD14-negative cells. Mol Microbiol. 39(3), 2001.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method comprising identifying an interaction between a pathogen and a biological agent, wherein identifying said interaction comprises providing a platform for supporting cell growth, said platform comprising a plurality of interaction sites, seeding said different interaction sites with different biological agents, said biological agents comprising cells, perfusing said platform with a fluid that carries substances for promoting growth and maintenance of said cells, exposing all of said interaction sites to a solution containing pathogens, and detecting evidence indicative of said interaction, said evidence comprising evidence indicative of a change in at least one of structure and composition of a medium at said interaction site.

2. The method of claim 1, further comprising selecting the pathogens to be viruses.

3. The method of claim 1, wherein said medium is an intracellular medium.

4. The method of claim 1, wherein said medium is an extracellular medium.

5. The method of claim 1, wherein seeding said different interaction sites of said platform with different biological agents comprises seeding said interaction sites with different kinds of cells.

6. The method of claim 1, wherein seeding said different interaction sites of said platform with different biological agents comprises seeding said interaction sites with different kinds of antibodies and the same kind of cell.

7. The method of claim 1, wherein seeding said different interaction sites of said platform with different biological agents comprises seeding said interaction sites with different kinds of antimicrobial agents and the same kind of cell.

8. The method of claim 1, wherein said pathogens comprise viruses, and wherein detecting evidence indicative of said interaction comprises detecting evidence indicative of replication of said viruses.

9. The method of claim 1, wherein said biological agents comprise antibodies, and wherein detecting evidence indicative of said interaction comprises detecting evidence indicative of said antibodies preventing infection of said cells.

10. The method of claim 1, wherein detecting evidence indicative of said interaction comprises detecting a change in metabolite level.

11. The method of claim 1, wherein detecting evidence indicative of said interaction comprises detecting a level of an extracellular compound.

12. The method of claim 1, wherein detecting evidence indicative of said interaction comprises detecting a level of an intracellular compound.

13. The method of claim 1, wherein detecting evidence indicative of said interaction comprises detecting a level of a compound that is depleted during the course of pathogen formation.

14. The method of claim 1, wherein detecting evidence indicative of said interaction comprises detecting a level of a compound that is synthesized during the course of pathogen formation.

15. The method of claim 1, wherein detecting evidence indicative of said interaction comprises detecting a glucose level.

16. The method of claim 1, wherein detecting evidence indicative of said interaction comprises detecting a change a change in ATP level.

17. The method of claim 1, wherein detecting evidence indicative of said interaction comprises detecting a change in at least one of pH and pOH.

18. The method of claim 1, wherein detecting evidence indicative of said interaction comprises detecting evidence of a redox reaction.

19. The method of claim 1, wherein detecting evidence indicative of said interaction comprises detecting a change in an ion concentration.

20. The method of claim 1, wherein detecting evidence indicative of said interaction comprises detecting a change in an hydroxide ion concentration.

21. The method of claim 1, wherein detecting evidence indicative of said interaction comprises detecting a change in a hydrogen ion concentration.

22. The method of claim 1, wherein detecting evidence indicative of said interaction comprises detecting a change in an electrical characteristic of said medium.

23. The method of claim 1, wherein detecting evidence indicative of said interaction comprises using dynamic light scattering to detect evidence of virus formation.

24. The method of claim 1, wherein detecting evidence indicative of said interaction comprises using an interferometer to detect evidence of virus formation.

25. The method of claim 1, wherein detecting evidence indicative of said interaction comprises using angle-resolved low-coherence interferometry to detect evidence of virus formation.

26. The method of claim 1, wherein said pathogen is a virus, and wherein said method further comprises, based on said interaction, identifying a host for said virus.

27. The method of claim 1, wherein said pathogen is a virus, and wherein said method further comprises, based on said interaction, identifying an antibody against said virus.

28. The method of claim 1, further comprising identifying an antimicrobial agent against said pathogen.

29. The method of claim 1, wherein seeding said different interaction sites of said platform with different biological agents comprises seeding at least one of said interaction sites with a plurality of antimicrobial agents and the same kind of cell.

30. The method of claim 29, further comprising identifying which of said antimicrobial agents is effective at blocking infection.

31. The method of claim 30, wherein identifying, which of said antimicrobial agents is effective at blocking infection comprises carrying out a binary search.

32. The method of claim 1, further comprising, after having identified said interaction, identifying a pathogen that engaged in said interaction and seeding a bioreactor with said identified pathogen.

33. The method of claim 1, wherein seeding said different interaction sites of said platform with different biological agents comprises seeding said interaction sites with different kinds of cells, identifying a kind of cell that functions as a host for said pathogen, and, after having identified said kind of cell, seeding all interaction sites with said kind of cell and with antimicrobial agents, wherein each interaction site is seeded with the same kind of cell but with a different antimicrobial agent, exposing said interaction sites to said pathogen, and, based on interactions between said pathogen and said interaction sites, identifying an antimicrobial agent that prevents infection of said kind of cell by said pathogen.

34. An apparatus comprising a platform, a fluid delivery system, and an activity-detector, wherein said fluid delivery system is coupled to said platform for providing an environment conducive to cell growth and maintenance on said platform, wherein said platform comprises interaction sites that are separated from each other, wherein said activity detector is optionally coupled to said platform, wherein said activity detector detects evidence of an interaction between a biological agent and a pathogen at said interaction site, said evidence comprising a change in a medium at said interaction site, said change being at least one of a change in structure and a change in composition.

35. The apparatus of claim 34, wherein said activity detector comprises an interferometer.

36. The apparatus of claim 34, wherein said activity-detector is configured to carry out angle-resolved, low-coherence interferometry.

37. The apparatus of claim 34, wherein said activity detector comprises an interferometer, and wherein said apparatus further comprises a processor that is configured to receive, from said interferometer, information representative of angular distribution of light that has been back-scattered from said interaction site, wherein said processor is further configured to recover, at least in part on the basis of said angular distribution, structural information about subsurface layers.

38. The apparatus of claim 34, wherein said activity detector is configured to provide data representative of dynamically scattered light and to provide said data to a processor, wherein said processor is further configured to recover, at least in part on the basis of said data, information indicative of a change in structure at said interaction site.

39. The apparatus of claim 34, wherein said activity detector is configured to detect a change in a concentration of metabolite at said interaction site said change in concentration being indicative of said interaction.

40. The apparatus of claim 34, wherein said activity detector is configured to detect a change, at said interaction site, of a concentration of a substance, said change in concentration being indicative of said interaction.

41. The apparatus of claim 34, wherein said activity detector is configured to detect a change in glucose levels at said interaction site, said change being indicative of said interaction.

42. The apparatus of claim 34, wherein said activity detector is configured to detect a change in an ion concentration at said interaction site, said change being indicative of said interaction.

43. The apparatus of claim 34, wherein said activity detector is configured to detect evidence of a redox reaction at said interaction site.

44. The apparatus of claim 34, wherein said activity detector is configured to detect a change in an electrical property of a medium at said interaction site.

45. A method comprising identifying an interaction between a toxin and a cell, wherein identifying said interaction comprises providing a platform for supporting cell growth, said platform comprising a plurality of interaction sites, seeding said different interaction sites with different biological agents comprising cells, perfusing said platform with a fluid that carries substances for promoting growth and maintenance of said cells, exposing all of said interaction sites to a solution containing the toxin, and detecting evidence indicative of said interaction, said evidence comprising evidence indicative of a change in at least one of structure and composition of a medium at said interaction site.
